(19) United States
(12) Patent Application Publication
(10) Pub. No.: US 2026/0224788 A1
(43) Pub. Date: Aug. 6, 2026

(54) BAG SYSTEM FOR PERITONAEAL DIALYSIS

(71) Applicant: Fresenius Medical Care Deutschland GmbH, Bad Homburg (DE)

(72) Inventors: Robert Berlich, Bad Homburg (GE); Pascal Joachimsky, Bad Homburg (DE); Michael Junker, Bad Homburg (DE); Andreas Klemm, Bad Homburg (DE); Franz Kugelmann, Bad Homburg (DE); Andreas Meiser; Tim Nikolaus, Bad Homburg (DE); Christian Schley, Bad Homburg (DE); Tobias Weber, Bad Homburg (DE)

(21) Appl. No.: 19/153,254

(22) PCT Filed: Jan. 31, 2024
(86) PCT No.: PCT/EP2024/052384

§ 371 Date: Aug. 1, 2025

(30) Foreign Application Priority Data
Feb. 2, 2023 (DE) 10 2023 102 599.6

(51) Int. Cl. A61M 1/28; A61J 1/10; A61M 1/16; B29C 65/00; B29C 65/02; B29L 31/00

(52) U.S. Cl. CPC A61M 1/28; A61J 1/10; A61M 1/1668; B29C 65/02; B29C 66/4322; A61M 2207/00; B29L 2031/7148

(57) ABSTRACT

The present disclosure relates to a bag system for peritoneal dialysis, including a solution bag and a drainage bag which are fluidically connected to each other (e.g., via a three-way valve), wherein the solution bag and the drainage bag each comprise one side formed by a folded-over film on which at least one connection is arranged using butt welding. The present disclosure further relates to a method for producing a bag system of this type.

### CROSS-REFERENCE TO RELATED APPLICATIONS

The present application is the national stage entry of International Patent Application No. PCT/EP2024/052384, filed Jan. 31, 2024, and claims priority to Application No. DE 102023102599.6, filed in the Federal Republic of Germany on Feb. 2, 2023, the contents of which are hereby incorporated by reference.

### TECHNICAL FIELD

The present disclosure relates to a bag system for peritoneal dialysis and to a method of producing such a bag system.

### BACKGROUND

Bag systems for peritoneal dialysis are known from the prior art, each having a solution bag with fresh dialysate and a drainage bag for collecting used dialysate. The solution bag and the drainage bag are connected to a three-way valve by means of a connection and a hose set.

The connection of the tube set to the respective bag traditionally requires a rather complicated structure, in which, for example, connectors with a special cross-sectional geometry, which are intended to allow the film to lie flush against the connector and thus ensure a tight seal, are glued or welded between two film layers of a bag.

If a bag that is to be used as a solution bag is finished, the bag is conventionally filled with ready-to-use dialysate, for example, via a filling port specially configured for this purpose.

This procedure increases the number of individual parts required for the bag system and is also associated with a high time expenditure for filling the bag through the filling port.

### SUMMARY

An advantage of the present disclosure is to mitigate or even completely eliminate the problems of the prior art. For example, an advantage of the present disclosure is to provide an inexpensive and easy-to-fill bag system for peritoneal dialysis.

Accordingly, a bag system for peritoneal dialysis is provided, comprising a solution bag and a drainage bag which are fluidically connected to each other (e.g., via a three-way valve). The solution bag and/or the drainage bag also each comprise one side formed by a folded-over film, on which at least one connection is arranged by means of butt welding.

Attaching the connection to the side formed by a folded-over film can provide the advantage that the connection sits on an outermost layer of the folded-over film and is not integrated into the folded-over film or its layer structure or interposed between two layers of film forming a bag.

In some examples, the term butt welding refers to a method in which two end faces are butt welded together in contact with each other. Butt welding is also known as mirror welding.

For example, the butt-welded connection can have a tube section. Firstly, the tube section is widened (e.g., in a tulip shape) at one end of the tube section in order to create a larger end face in the manner of a flange. The end face of the tube section is then placed on a surface, such as a film, and welded thereto.

In this procedure, the end face of the tube section then sits on the surface of the film and is not integrated into it, for example in the case of a multi-layer film it is arranged between the layers of the multi-layer film or interposed between two films.

Butt welding can be used to create strong and resilient joints.

A plurality of connections may be arranged on the side of the bag formed by a folded-over film, at least one (e.g., all) of which are arranged on the film by means of butt welding.

According to one embodiment, the solution bag and the drainage bag are identically configured. This enables a simple design of the bag system. In some embodiments, neither the solution bag nor the drainage bag has a filling port in addition to the respective butt-welded connection.

It is further conceivable that the solution bag and/or the drainage bag each comprises a respective sealing seam on a side opposite the connection, e.g., with one or more suspension eyelet(s). In some implementations, the width of the sealing seam is such that the suspension eye(s) is/are completely surrounded by the sealing seam.

According to one embodiment, the solution bag and/or the drainage bag has a receiving area bounded in three sides by a sealing seam and on one side by a folded-over film.

Before the bag system is used for peritoneal dialysis, the collection area of the solution bag can be filled with ready-to-use dialysate and the collection area of the drainage bag is empty. At the start of a peritoneal dialysis treatment cycle, the used dialysis solution from the previous treatment cycle is drained from the patient's peritoneal cavity into the drainage bag. The contents of the solution bag are then transferred to the patient's peritoneal cavity.

According to one embodiment, at least the solution bag and also the drainage bag does not have a filling port for filling a receiving area of the bag (e.g., in the form of a connection, port, or connector).

Such a filling port can be understood as an access to a receiving area of the bag, which is configured to fill the receiving area of the bag before the bag or bag system is delivered to an end user or before the bag is used in peritoneal dialysis. After filling, the filling port is usually closed permanently, for example with a sealing cap.

According to one embodiment, the connection of the solution bag and/or drainage bag is welded on in such a way that it sits on an outermost layer of the folded-over film and is not integrated into the folded-over film, two folded-over layers of the film or a layer structure of the film.

For example, the connection can consist of a tube section or comprise a tube section. In some embodiments, the tube section forms the connection so that the tube section is butt-welded directly onto the film of the bag.

In the area of the connection, the bag can have an opening in the film through which the content can flow out of the bag's receiving area.

According to one embodiment, the solution bag, and the drainage bag as well as a tube set fluidically connecting these bags are made of a polymer material and can be configured as single-use articles or disposable.

Another aspect of the present disclosure relates to the use of a bag system as described herein for peritoneal dialysis.

Furthermore, the present disclosure relates to a method of producing a bag system, the method including:
a) providing a flat film with two outer edges, wherein the flat film can be a multi-layer film or a single-layer film;
b) fixing at least one connection to the flat film by means of butt welding, and, e.g., producing an opening of the flat film in the region of the connection, for example by punching out or using a hot mandrel;
c) producing a film tube out of the flat film, e.g., by means of a forming shoulder and by sealing the outer edges of the flat film together;
d) producing a sealing seam that extends perpendicular or substantially perpendicular to a longitudinal direction of the film tube, whereby a receiving pocket is produced in the film tube;
e) optionally filling the receiving pocket along the longitudinal direction of the film tube with a medical solution (e.g., ready-to-use dialysate); and
f) closing the receiving pocket by producing an additional second sealing seam that extends perpendicular or substantially perpendicular to the longitudinal direction of the film tube, which second sealing seam is spaced apart from the first sealing seam in the longitudinal direction of the film tube, whereby the receiving pocket is closed, and a bag is formed.

A separation then takes place along the first and second sealing seams, which run transversely to the longitudinal direction of the film tube, whereby an individual bag is separated from the film tube.

Two such bags are then used in a bag system according to the disclosure, wherein one of the bags has been filled according to step e) and is used as a solution bag and the other of the bags is initially empty and is used as a drainage bag.

Thus, in other words, to produce a bag system according to the disclosure, e.g., two bags produced by means of a method according to the disclosure are provided, wherein one bag is filled according to step e) and the other bag is empty and each of the bags are connected with their butt-welded connection to a common three-way valve. Filling can be carried out vertically from above.

According to one embodiment of a method according to the disclosure, bags are produced continuously along a continuous film tube, in each case connected to each other via a sealing seam, which bags are separated from each other by separation along a sealing seam extending perpendicular or substantially perpendicular to the longitudinal direction of the film tube.

The following parameters can be used for butt welding in step b):

| | |
|---|---|
| Welding temperature: | 150° C. ≤ T ≤ 300° C. |
| Welding time: | 0.7 s ≤ t ≤ 2 s |
| Welding pressure: | 0.1N/mm² ≤ σ ≤ 2N/mm² |

The following parameters can be used:

| | |
|---|---|
| Welding temperature: | 190° C. ≤ T ≤ 250° C. |
| Welding time: | 0.9 s ≤ t ≤ 1.5 s |

Welding pressure: 3000N where A = 6300 mm² → 0.48N/mm²

In some embodiments, bags are produced continuously along a continuous film tube, in each case connected to each other via a sealing seam, which bags are separated from each other by separation along a transverse sealing seam extending perpendicular or substantially perpendicular to the longitudinal direction of the film tube.

In other words, a continuous film tube can be produced, which, for example, is filled in the longitudinal direction of the tube with ready-to-use dialysate, for example and which is portioned into different bags by sealing in the transverse direction.

Thus, for example, when the tube is filled, a pillar of filling material is produced, which is divided into several bags by transverse sealing, which can serve as solution bags in a bag system.

In some embodiments, the method according to the present disclosure can be carried out with a single machine.

According to one embodiment of the method, the filled bags and the unfilled drainage bags are produced on separate production lines and then combined to form the bag system. For example, the filled solution bags are produced on a first machine while the drainage bags are produced on a second machine.

At this point, it should be noted that the present disclosure is not limited to the explicitly disclosed combinations of features, but that the features disclosed herein may also be claimed in other combinations or in isolation.

Further advantages, effects, and features of the present invention are shown in the following description of embodiments with reference to the drawings, in which identical reference symbols denote identical or similar components.

### BRIEF DESCRIPTION OF THE FIGURES

FIG. 1 shows a conventional bag system for peritoneal dialysis;

FIG. 2 shows a bag system for peritoneal dialysis; and

FIG. 3 shows continuous production of bags from a continuous film tube, which can be used in the context of the present disclosure.

### DETAILED DESCRIPTION

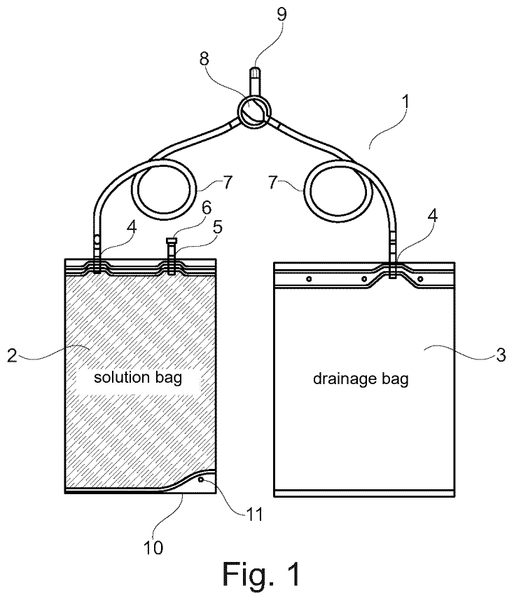

FIG. 1 shows a bag system 1 for peritoneal dialysis with a bag 2 (also referred to as a solution bag) filled with a solution, for example with ready-to-use dialysate, and an empty bag 3 (also referred to as a drainage bag), for example for holding used dialysate.

The bags 2 and 3 each have a connector or connection 4, which is arranged between two layers of film of the bag welded together in the area of the connection 4. In other words, each connection 4 is arranged between the film layers forming the bag 2 or 3 and is enclosed by them.

The bag 2 also has a filling port 5, which is non-detachably closed with a sealing cap 6. The filling port 5 is used to fill the bag 2 with solution, for example dialysate.

Once the bag 2 has been filled to the desired volume, the filling port 5 is permanently closed with the sealing cap 6 in such a way that an end user cannot open the filling port 5.

The bags 2 and 3 are each fluidically connectable or connected to a three-way valve 8 via a tube 7 or a line. The three-way valve 8 can also be connected to a patient connector 9.

Using the three-way valve 8, fresh dialysate from the bag 2 can be fed to a patient via the patient connector 9 and used dialysate from the patient can be fed into the drainage bag 3 via the patient connector 9.

The bag 2 can be positioned in an elevated position relative to the patient, for example in a gravimetric peritoneal dialysis system, so that the dialysate flows from the bag 2 to the patient connector 9 due to gravity.

For this purpose, the bag 2 also has a suspension area 10 with a suspension eye 11.

Disadvantages of a conventional bag system shown in FIG. 1 include:
the connections 4 arranged between the foils of the respective bag 2,3, which are associated with high production costs,
the need for a filling port 5 to fill the bag 2, and
filling the bag 2 via the filling port 5.

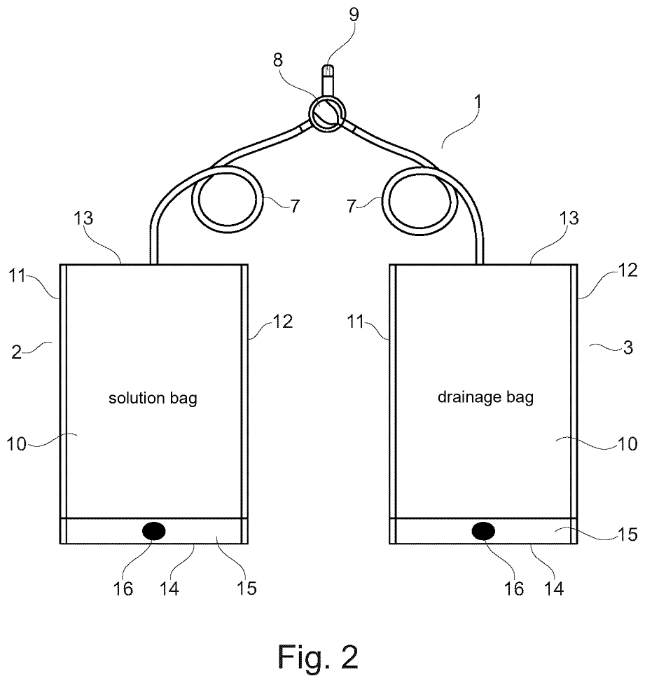

FIG. 2 shows a bag system 1 for peritoneal dialysis according to the present disclosure with a bag 2 (also referred to as a solution bag) filled with a solution, for example with ready-to-use dialysate, and an empty bag 3 (also referred to as a drainage bag), for example for holding used dialysate.

According to the present embodiment, the solution bag 2 and the drainage bag 3 can have an identical structure.

The bags 2 and 3 each have a receiving area 10, which case of the solution bag 2 can be filled with fresh dialysate and in the case of the drainage bag is initially empty but can be successively filled with used dialysate.

The receiving area 10 of each bag 2, 3 is bounded on a first and a second opposite side 11 and 12 by a sealing seam.

Each of the bags 2, 3 also has a third side 13 formed by a folded-over film, on which at least one connection 7 is non-detachably arranged by means of butt welding.

Opposite the third side 13, each of the bags 2, 3 has a fourth side 14 with a sealing seam 15, in which at least one suspension eye 16 is provided.

Each of the bags 2, 3 can be produced from a flat film by folding the film over on the third side 13 and sealing the outer edges of the flat film together on the fourth side 14 to form the sealing seam 15, which initially creates a film tube.

A transverse sealing seam is then formed transversely to a longitudinal direction of the film tube, which delimits the first side 11 of each bag 2, 3.

In this intermediate state, the receiving area 10 is still open on its side opposite the first side 11 of each bag 2, 3 and can thus be easily filled if desired. The bag can also remain empty.

This eliminates the need for a filling port 5 on the solution bag 2.

After filling the receiving area 10, the transverse sealing seam provided on the second side 12 of the bag is created, whereby the receiving area 10 of each bag 2, 3 is closed.

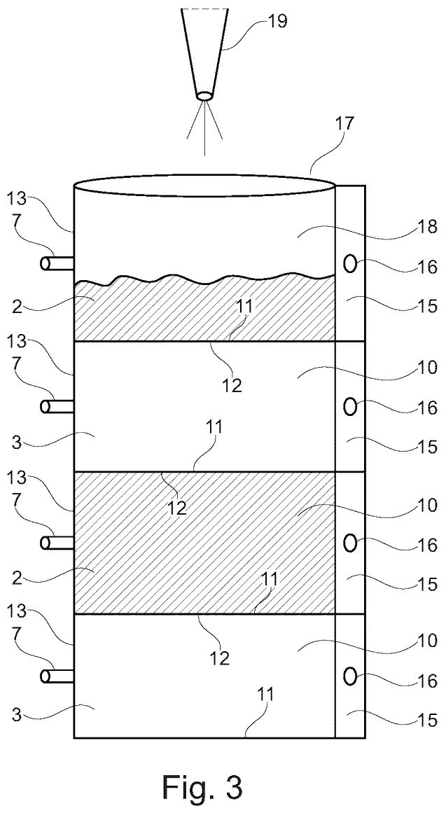

FIG. 3 shows, by way of example, continuous production of bags from a continuous film tube, which can be used in the context of the present disclosure.

The film tube 17 shown in FIG. 3 can extend in a vertical direction during the filling process shown in FIG. 3.

The film tube 17 was produced from a flat film by folding the film over and sealing the outer edges of the flat film together in the sealing seam 15.

In the view shown in FIG. 3, the film tube 17 comprises four bags, namely two solution bags 2 (one of which is already filled and sealed and one of which is being filled) and two drainage bags 3 (the receiving area 10 of which is empty).

The solution bags 2 and drainage bags 3 are arranged alternately in the longitudinal direction of the film tube 17.

However, this arrangement is merely exemplary; it is also conceivable to provide only solution bags 2 or only drainage bags 3 within a film tube 17. If the solution bag 2 and drainage bag 3 are provided in the frame of a film tube 17, their arrangement relative to each other is arbitrary.

In the view of the film tube 17 shown in FIG. 3, the bags 2 and 3 are not yet separated from each other, but are each connected to each other by a common transverse sealing seam on the sides 11, 12.

The folded-over side of the flat film forms the third side 13 (left in FIG. 3) of each bag 2, 3 and the sealing seam 15 forms the fourth side 14 of each bag 2, 3.

Each bag 2, 3 is equipped with at least one butt-welded connection 7 on its third side 13.

The uppermost bag shown in FIG. 3 illustrates the filling of the receiving area 10 of a solution bag 2. In this bag 2, the receiving area 10 is not yet closed or sealed at its upper end shown in FIG. 2 and therefore forms a receiving pocket 18 for filling material.

The receiving pocket 18 is filled with a specific content, for example the dialysate indicated by hatching, e.g., from above in the vertical direction by means of a separate filling device, for example a filling lance 19. Once the receiving pocket 18 is sufficiently filled, it is closed by transverse sealing.

This type of filling eliminates the need to equip each solution bag 2 with a filling port 5. In addition, the filling speed can be increased compared to conventional filling using a filling port 5.

To separate the individual bags 2, 3 of the continuous film tube 17 from each other, they are cut apart in the transverse direction along the common transverse sealing seam on the sides 11, 12.

The bags 2 and 3 can now be used as part of a bag system.

1-10. (canceled)

11. A bag system for peritoneal dialysis, the bag system comprising:
a solution bag; and
a drainage bag, wherein:
the solution bag and the drainage bag are fluidically connected to each other; and
the solution bag and/or the drainage bag comprises one side formed by a folded-over film, on which at least one connection is arranged by butt welding.

12. The bag system of claim 11, wherein the solution bag and the drainage bag are fluidically connected to each other via a three-way valve.

13. The bag system of claim 11, wherein the solution bag and the drainage bag are of identical design.

14. The bag system of claim 11, wherein the solution bag and/or the drainage bag comprises a respective sealing seam with a suspension eyelet on a side opposite the connection.

15. The bag system of claim 11, wherein the solution bag and/or the drainage bag comprises a receiving area bounded on three sides by a sealing seam and on one side by the folded-over film.

16. The bag system of claim 11, wherein at least the solution bag does not have a filling port for filling a receiving area of the solution bag.

17. The bag system of claim 16, wherein the drainage bag does not have a filling port for filling a receiving area of the drainage bag.

18. The bag system of claim 16, wherein the solution bag does not have a connection, port, or connector for filling the receiving area of the solution bag.

19. The bag system of claim 11, wherein the at least one connection of the solution bag and/or drainage bag is welded in such a way that the at least one connection sits on an outermost layer of the folded-over film and is not integrated into the folded-over film, two folded-over layers of the film, or a layer structure of the film.

20. The bag system of claim 11, wherein the solution bag, the drainage bag, and a tube set fluidically connecting the solution bag and the drainage bag are made of a polymer material.

21. The bag system of claim 20, wherein the solution bag, the drainage bag, and the tube set are configured as disposable articles.

22. A method for producing a bag system, the method comprising:
providing a flat film with two outer edges;
fixing at least one connection to the flat film using butt welding;
producing a film tube out of the flat film by sealing the outer edges of the flat film together;
producing a first sealing seam that extends perpendicular or substantially perpendicular to a longitudinal direction of the film tube, whereby a receiving pocket is produced in the film tube; and
closing the receiving pocket by producing a second sealing seam that extends perpendicular to the longitudinal direction of the film tube, wherein the second sealing seam is spaced apart from the first sealing seam in the longitudinal direction of the film tube, whereby the receiving pocket is closed and a bag is formed.

23. The method of claim 22, further comprising filling the receiving pocket along the longitudinal direction of the film tube with a medical solution.

24. The method of claim 23, wherein the medical solution comprises ready-to-use dialysate.

25. The method of claim 22, wherein multiple bags are produced continuously along a continuous film tube, wherein the multiple bags are connected to each other by the sealing seam, and the multiple bags are separated from each other by separation along the sealing seam extending perpendicular to the longitudinal direction of the film tube.

26. The method of claim 25, wherein two bags are produced, wherein one bag is filled and the other bag is empty, and wherein each of the bags are connected with a butt-welded connection to a common three-way valve.

27. The method of claim 22, further comprising forming a suspension eyelet along the second sealing seam.

28. The method of claim 22, wherein the at least one connection is welded such that the at least one connection sits on an outermost layer of a folded-over film and is not integrated into the folded-over film, two folded-over layers of the folded-over film, or a layer structure of the folded-over film.

29. The method of claim 22, wherein the butt welding comprises a welding temperature between 150° C. and 300° C.

30. The method of claim 22, wherein the butt welding comprises a welding time in a range of 0.7 s to 2 s.
